(19) United States
(12) Patent Application Publication
(10) Pub. No.: US 2012/0265159 A1
(43) Pub. Date: Oct. 18, 2012

(54) DEVICE FOR STORING AND DISPENSING A MEDICAMENT

(75) Inventor: Alex M. Kaufman, Dresher, PA (US)

(73) Assignee: VELCERA, INC., Yardley, PA (US)

(21) Appl. No.: 13/085,022

(22) Filed: Apr. 12, 2011

(51) Int. Cl. A61M 35/00

(52) U.S. Cl. 604/310

(57) ABSTRACT

An apparatus includes a substrate and a wall coupled to the substrate. The substrate and the wall define a medicament reservoir. At least a portion of the wall is configured to be deformed to reduce a volume of the medicament reservoir when the wall is actuated. The wall includes an actuation portion, which can be used to actuate the wall. The portion of the wall is configured to deform at a first rate when the actuation portion is in a first configuration, and a second rate when the actuation portion is in a second configuration.

### BACKGROUND

The embodiments described herein relate generally to a device for storing and dispensing medicaments, and more particularly to a device that can be used to dispense a parasiticidal medicament to the skin of an animal.

Some known medicament containers can be used to dispense a topical medicament contained therein. For example, some known medicament containers can contain a topical flea and/or tick preventative formulation, and can include a tip through which the formulation can be dispensed onto the skin of an animal. Some known medicament containers used for dispensing such formulations are constructed from a flexible material such that the medicament contained therein can be dispensed when a user squeezes or compresses a portion of the container.

Such known containers, however, can often result in improper dosage and/or delivery of the medicament. For example, some known containers are constructed from a material that is easily compressed, which can result in the delivery of a portion of the medicament contained therein even when a very slight compression force is applied. Conversely, some known containers require a greater compression force and thus do not reliably deliver the full dose of the medicament contained therein.

Thus, a need exists for an improved device for containing and dispensing a medicament.

### SUMMARY

Devices for containing and dispensing a medicament are described herein. In some embodiments, an apparatus includes a substrate and a wall coupled to the substrate. The substrate and the wall define a medicament reservoir. At least a portion of the wall is configured to be deformed to reduce a volume of the medicament reservoir when the wall is actuated. The wall includes an actuation portion, which can be used to actuate the wall. The actuation portion of the wall is configured to deform at a first rate when the actuation portion is in a first configuration, and a second rate when the actuation portion is in a second configuration.

### BRIEF DESCRIPTION OF THE DRAWINGS

FIGS. 1-3 are schematic illustrations of a medicament container according to an embodiment in a first configuration, a second configuration and a third configuration, respectively.

FIG. 4 is a graphical representation of the rate of deformation of a portion of the medicament container shown in FIGS. 1-3.

FIGS. 5 and 6 are schematic illustrations of a medicament container according to an embodiment in a first configuration and a second configuration, respectively.

FIGS. 7 and 8 are schematic illustrations of a medicament container according to an embodiment in a first configuration and a second configuration, respectively.

FIGS. 9 and 10 are perspective views of a medicament container according to an embodiment.

FIGS. 11-13 are a top view, a side view and a front view, respectively, of the medicament container shown in FIGS. 9 and 10.

FIGS. 14 and 15 show a portion of the medicament container identified as portion Z in FIG. 12, in a first configuration and a second configuration, respectively.

FIG. 16 shows a package according to an embodiment containing the medicament container shown in FIGS. 9 and 10.

FIGS. 17-20 are a perspective view, a top view, a side view and a front view, respectively, of a medicament container according to an embodiment.

FIGS. 21-24 are a perspective view, a top view, a side view and a front view, respectively, of a medicament container according to an embodiment.

FIGS. 25-28 are a perspective view, a top view, a side view and a front view, respectively, of a medicament container according to an embodiment.

FIGS. 29-32 are a perspective view, a top view, a side view and a front view, respectively, of a medicament container according to an embodiment.

### DETAILED DESCRIPTION

Devices for containing and dispensing a medicament are described herein. In some embodiments, an apparatus includes a substrate and a wall coupled to the substrate. The substrate and the wall define a medicament reservoir, which can contain, for example, a parasiticidal formulation to be delivered onto the skin of an animal. At least a portion of the wall is configured to be deformed to reduce a volume of the medicament reservoir when the wall is actuated. The wall includes an actuation portion, which can be used to actuate the wall. The portion of the wall is configured to deform at a first rate when the actuation portion is in a first configuration, and a second rate when the actuation portion is in a second configuration.

In some embodiments, an apparatus includes a medicament container including a container portion and a dispensing portion. The container portion includes a wall that defines, at least in part, a medicament reservoir. At least a portion of the wall is configured to be deformed to reduce a volume of the medicament reservoir. The wall includes a protrusion that extends from the medicament reservoir, and that defines a stress concentration riser configured to propagate deformation of the wall from a predetermined location of the wall. The dispensing portion is configured to place the medicament reservoir in fluid communication with a volume outside of the medicament container. In this manner, a medicament contained within the medicament reservoir can be delivered via the dispensing portion when the volume of the medicament reservoir is reduced.

In some embodiments, an apparatus includes a first layer and a second layer. The second layer is coupled to the first layer such that the first layer and the second layer define a medicament reservoir. At least a portion of the second layer is configured to be deformed to reduce a volume of the medicament reservoir when the second layer is actuated. The second layer is tapered such that a cross-sectional area of the medicament reservoir at a first location along a center line of the medicament reservoir is greater than a cross-sectional area of the medicament reservoir at a second location along the center line. The second layer includes an actuation portion configured to propagate deformation of the second layer from the first location towards the second location.

As used in this specification, the words “proximal” and “distal” refer to the direction closer to and away from, respectively, a user who would place the device into contact with a patient and/or an animal. Thus, for example, the end of a device first touching the body of the patient and/or the animal would be the distal end, while the opposite end of the device (e.g., the end of the device being manipulated by the user) would be the proximal end of the device.

As used herein, the term “stiffness” relates to an object's resistance to deflection, deformation, and/or displacement by an applied force. For example, a wall of a container with greater stiffness is more resistant to deflection, deformation and/or displacement when exposed to a force than a wall of a container having a lower stiffness. Similarly stated, a container having a higher stiffness can be characterized as being more rigid than a container having a lower stiffness. Stiffness can be characterized in terms of the amount of force applied to the object and the resulting distance through which a first portion of the object deflects, deforms, and/or displaces with respect to a second portion of the object. This can be depicted graphically as a stress-strain curve. When characterizing the stiffness of an object, the deflected distance may be measured as the deflection of a portion of the object different than the portion of the object to which the force is directly applied. Said another way, in some objects, the point of deflection is distinct from the point where force is applied.

Stiffness is an extensive property of the object being described, and thus is dependent upon the material from which the object is formed as well as certain physical characteristics of the object (e.g., shape and boundary conditions). For example, the stiffness of an object can be increased or decreased by selectively including in the object a material having a desired modulus of elasticity, flexural modulus and/or hardness. The modulus of elasticity is an intensive property of (i.e., is intrinsic to) the constituent material and describes an object's tendency to elastically (i.e., non-permanently) deform in response to an applied force. A material having a high modulus of elasticity will not deflect as much as a material having a low modulus of elasticity in the presence of an equally applied stress. Thus, the stiffness of the object can be increased, for example, by introducing into the object and/or constructing the object of a material having a high modulus of elasticity.

Similarly, the flexural modulus is used to describe the ratio of the applied stress on an object in flexure to the corresponding strain in the outermost portions of the object. The flexural modulus, rather than the modulus of elasticity, is used to characterize certain materials, for example plastics, that do not have material properties that are substantially linear over a range of conditions. An object with a first flexural modulus is less elastic and has a greater strain on the outermost portions of the object than an object with a second flexural modulus lower than the first flexural modulus. Thus, the stiffness of an object can be increased by including in the object a material having a high flexural modulus.

The hardness of a material describes an object's tendency to plastically (i.e., permanently) deform in response to an applied force. The hardness of a material can be dependent on more than one intensive property of a material, such as for example, the ductility, the material toughness and/or the elasticity (e.g., as characterized by the modulus of elasticity). The hardness of a material may be characterized as its “durometer,” in reference to the apparatus used to measure the hardness of the types of material often used to form the medicament containers disclosed herein. Thus, for example, an object with a first durometer is less elastic and has a greater strain on the outermost portions of the object than an object with a second flexural modulus lower than the first flexural modulus. Thus, an object constructed from a material having a high durometer will not deflect as much as a material having a low durometer in the presence of an equally applied stress. Thus, the stiffness of the object can be increased, for example, by introducing into the object and/or constructing the object of a material having a high durometer.

The stiffness of an object can also be increased or decreased by changing a physical characteristic of the object, such as the shape or cross-sectional area of the object. For example, an object having a length and a cross-sectional area may have a greater stiffness than an object having an identical length but a smaller cross-sectional area. As another example, the stiffness of an object can be reduced by including one or more stress concentration risers (or discontinuous boundaries) that cause deformation to occur under a lower stress and/or at a particular location of the object. Thus, the stiffness of the object can be increased by increasing and/or changing the shape of the object.

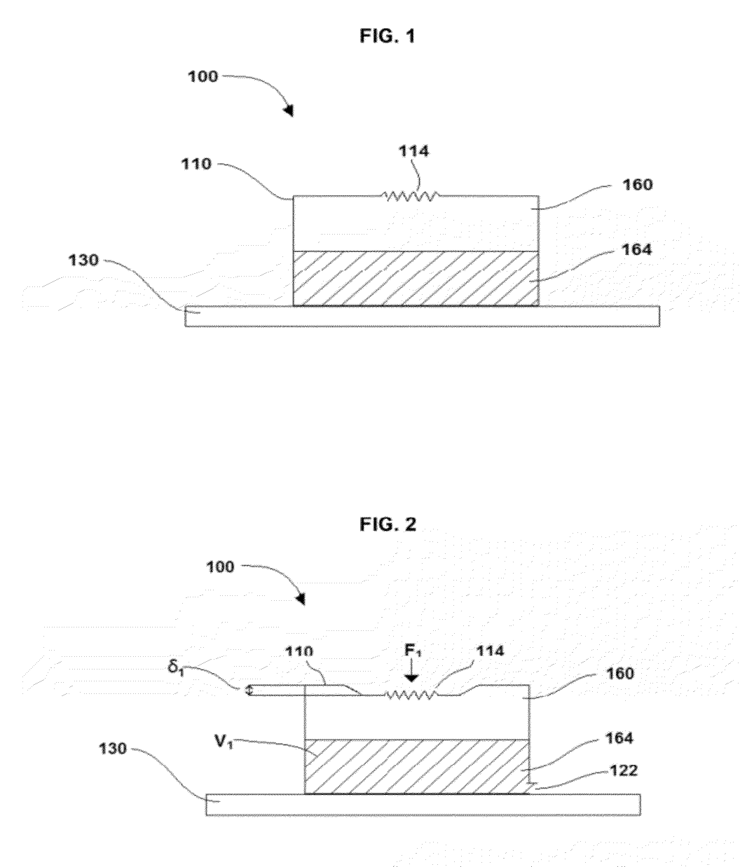

FIGS. 1-3 are schematic illustrations of a medicament container 100 according to an embodiment in a first configuration, a second configuration and a third configuration, respectively. The medicament container 100 includes a substrate 130 and a wall 110 coupled to the substrate 130. The wall 110 defines a medicament reservoir 160 within which a medicament 164 can be disposed. The medicament 164 can be any suitable medicament, such as for example, a parasiticidal formula to be topically applied to an animal. In some embodiments, for example, the medicament 164 can be formulated to contain fipronil or a veterinarily acceptable derivative thereof. In some embodiments, the medicament 164 can be any of the formulations disclosed in U.S. Patent Publication No. 2011/0060023, entitled “Parasiticidal Formulation,” filed Mar. 18, 2010, which is incorporated herein by reference in its entirety.

Although the medicament reservoir 160 is shown as being only partially filled with the medicament 164, in other embodiments, the medicament reservoir 160 can be substantially entirely filled with the medicament 164. Similarly stated, in some embodiments, the volume of the medicament 164 when the medicament container 100 is in the first (or storage) configuration, as shown in FIG. 1, is substantially the same as the volume of the medicament reservoir 160 defined by the wall 110.

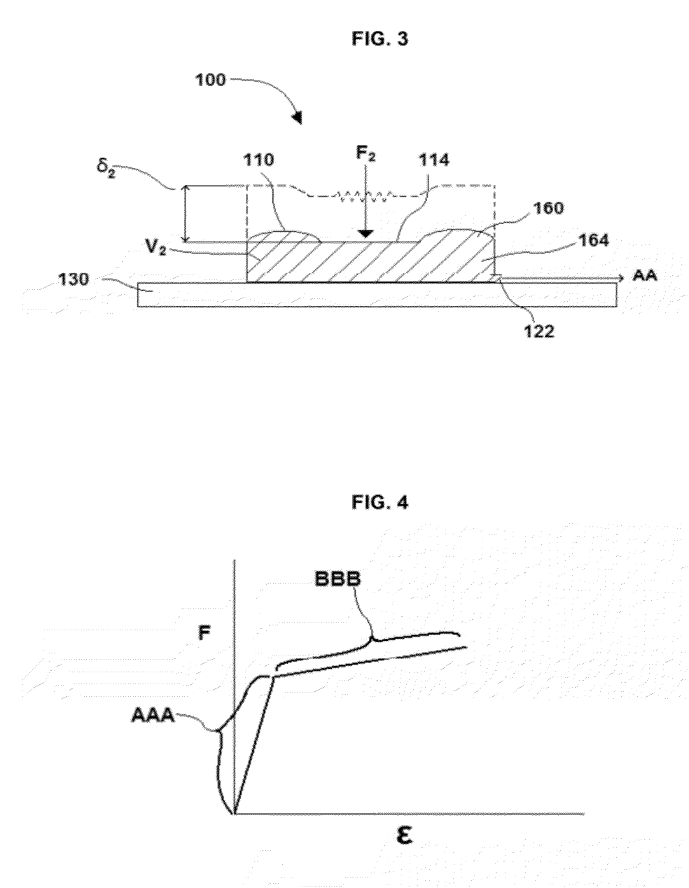

The wall 110 includes an actuation portion 114, and can define an opening 122 (see, e.g., FIGS. 2 and 3) through which the medicament 164 can be conveyed. The opening 122 can be defined by any suitable mechanism, such as, for example, by puncturing a portion of the wall 110, by removing a portion of the wall 110, by removing a cap, plug, seal or other structure from the wall 110, or the like. As shown in FIGS. 2 and 3, the wall 110 and/or the medicament container 100 can be actuated when a force (e.g., the force F₁ and/or the force F₂) is applied to the actuation portion 114 of the wall 110. In this manner, the medicament 164 can be conveyed and/or delivered from the medicament reservoir 160 to a volume outside of the medicament container 100, as described in more detail herein.

As shown in FIGS. 2 and 3, when the wall 110 and/or the medicament container 110 is actuated, at least a portion of the wall 110 is deformed and/or displaced, thereby reducing the volume of the medicament reservoir 160. In this manner, the medicament 164 can be conveyed from the medicament reservoir 160 in response to the change in the volume of the medicament reservoir 160. Similarly stated, when the wall 110 is deformed and/or displaced, at least a portion of the force applied to the actuation portion 114 acts upon the medicament 164 thereby causing the medicament to flow out of the medicament reservoir 160. The operation of the medicament container 100 is described below with reference to FIGS. 1-3, which show the medicament container 100 in three different configurations, and FIG. 4, which graphically represents the rate of deformation of a portion of the wall 110 when the medicament container 100 and/or the wall 110 is actuated.

More particularly, in use, the medicament container 100 can be moved between a first (or storage) configuration (see FIG. 1), a second (or initial actuation) configuration (see FIG. 2) and a third (or full actuation) configuration (FIG. 3). When the medicament container 100 is in the first configuration, the medicament reservoir 160 is fluidically isolated from the volume outside of the medicament container 100. Similarly stated, the substrate 130 and the wall 110 collectively define a substantially hermetic and/or fluid-tight seal to prevent leakage of the medicament 164 from the medicament reservoir 160. As shown in FIG. 1, the actuation portion 114 is in its first configuration when the medicament container 100 is in its first configuration.

The medicament container 100 is moved from its first configuration (FIG. 1) to its second configuration (FIG. 2) when the opening 122 is defined by the wall 110, and a force F₁ is applied to the actuation portion 114 of the wall 110. As shown in FIG. 2, the actuation portion 114 remains in its first configuration when the medicament container 100 is in its second configuration. Similarly stated, the force F₁ is not sufficient to move the actuation portion from its first configuration (FIGS. 1 and 2) to its second configuration (FIG. 3). The application of the force F₁, however, causes a portion of the wall 110 to deform, deflect and/or be displaced by a distance δ₁. The deformation, displacement and/or deflection of the portion of the wall 110 is graphically represented in FIG. 4, which shows a stress-strain curve for the portion of the wall 110. In particular, the x-axis represents the strain of the portion of the wall 110 (which is associated with the deformation, deflection and/or displacement of the portion of the wall 110) and the y-axis represents the stress applied to the actuation portion 114 of the wall 110 (which is associated with the force applied to the actuation portion 114). The region of the graph identified as region AAA corresponds to the deformation, deflection and/or displacement of the portion of the wall 110 when the medicament container 100 is in its second configuration and the actuation portion 114 is in its first configuration.

As shown in FIG. 4, the portion of the wall 110 deforms, deflects and/or is displaced at a first rate when the actuation portion 114 is in its first configuration (and the medicament container is in its second configuration). Although the first rate of deformation, which is the slope of the stress-strain line in region AAA, is shown as being substantially constant, in other embodiments, the first rate of deformation can vary within the region AAA. Similarly stated, although the stress-strain line in region AAA is shown as being substantially linear, in other embodiments, the stress-strain line in region AAA can be non-linear.

The deformation, displacement and/or deflection of the portion of the wall 110 when the actuation portion 114 is in its first configuration (and the medicament container is in its second configuration) reduces the volume of the medicament reservoir 160 to the volume V₁, as shown in FIG. 2. Although the medicament 164 is shown as remaining within the medicament reservoir 160 when the medicament container 100 is moved from its first configuration to its second configuration, in other embodiments, a portion of the medicament 164 can be conveyed from the medicament reservoir 160 via the opening 122 when the portion of the wall 110 is deformed, displaced and/or deflected as shown in FIG. 2.

The medicament container 100 is moved from its second configuration (FIG. 2) to its third configuration (FIG. 3) when a force F₂, which is greater than the force F₁, is applied to the actuation portion 114 of the wall 110. As shown in FIG. 3, the application of the force F₂ causes the actuation portion 114 to move from its first to its second configuration. Thus, when the actuation portion 114 is moved from its first configuration (FIGS. 1 and 2) to its second configuration (FIG. 3), the medicament container 100 is moved from its second configuration to its third configuration. In some embodiments, the application of the force F₂ causes the actuation portion 114 to substantially suddenly and/or discontinuously move from its first configuration to its second configuration. In some embodiments, for example, the actuation portion 114 can include a stress concentration riser (e.g., a discontinuous boundary, a region of reduced thickness or the like, not shown in FIGS. 1-3) to promote the sudden and/or discontinuous movement of the actuation portion 114 from its first configuration to its second configuration. In some embodiments, the actuation portion 114 can include a detent mechanism (not shown in FIGS. 1-3) to promote the sudden and/or discontinuous movement of the actuation portion 114 from its first configuration to its second configuration.

Moreover, the application of the force F₂ also causes a portion of the wall 110 to deform, deflect and/or be displaced by a distance δ₂. The deformation, displacement and/or deflection of the portion of the wall 110 when the actuation portion 114 is in its second configuration (and the medicament container is in its third configuration) reduces the volume of the medicament reservoir 160 to the volume V₂, as shown in FIG. 3. The reduction in the volume of the medicament reservoir 160 results in at least a portion of the medicament 164 being conveyed and/or dispensed from the medicament reservoir 160 via the opening 122, as shown by the arrow AA in FIG. 3.

The deformation, displacement and/or deflection of the portion of the wall 110 when the medicament container 100 is in its third configuration and the actuation portion 114 is in its second configuration is identified as region BBB in the graph shown in FIG. 4. As shown in FIG. 4, the portion of the wall 110 deforms, deflects and/or is displaced at a second rate (different from the first rate) when the actuation portion 114 is in its second configuration (and the medicament container is in its third configuration). By changing the rate of deformation, the pressure of the medicament 164 within the medicament reservoir 160 and/or the flow rate of the portion of the medicament 164 begin conveyed from the medicament reservoir 160 during actuation of the medicament container 100 can be controlled to a desired value. This arrangement can also result in a consistent delivery of a desired dose of the medicament 164 during actuation of the medicament container 100. Although the second rate of deformation is shown as being “higher” or “faster” than the first rate of deformation, in other embodiments, the second rate of deformation can be “lower” or “slower” than the first rate of deformation.

Although the second rate of deformation, which is the slope of the stress-strain line in region BBB, is shown as being substantially constant, in other embodiments, the second rate of deformation can vary within the region BBB. Similarly stated, although the stress-strain line in region BB is shown as being substantially linear, in other embodiments, the stress-strain line in region BBB can be non-linear. In embodiments in which the first rate of deformation and/or the second rate of deformation are non-linear, the transition of the actuation portion 114 from its first configuration to its second configuration produces a discontinuity between the portion of the curve representing the deformation of the portion of the wall 110 when the actuation portion 114 is in its first configuration and the portion of the curve representing the deformation of the portion of the wall 110 when the actuation portion 114 is in its second configuration.

Although the wall 110 is shown as being configured to define the opening 122, in other embodiments, the medicament container 100 can include a pipette and/or a second wall (not shown in FIGS. 1-3) that defines a lumen in fluid communication with the medicament reservoir 160, and through which the medicament 164 can be dispensed. Such arrangements can limit the contact between the user and the medicament 164. Moreover, such arrangements can direct the flow of the medicament 164 from the medicament reservoir in a predetermined direction.

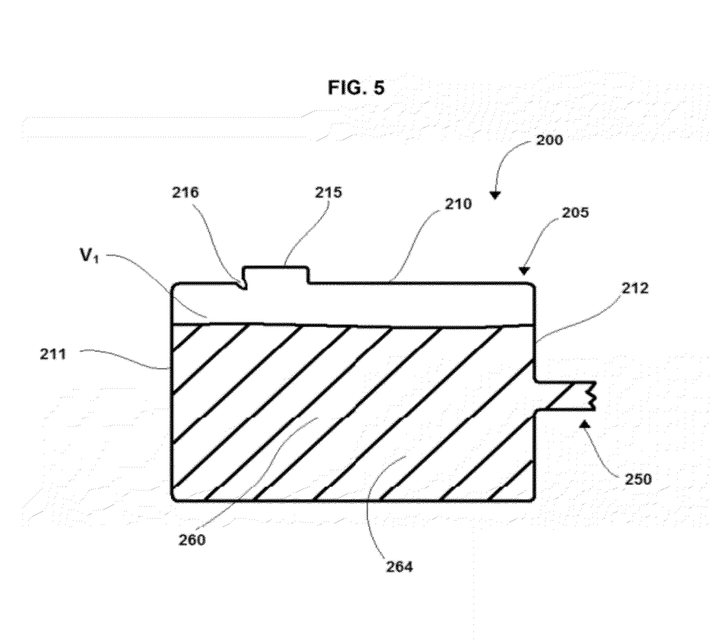

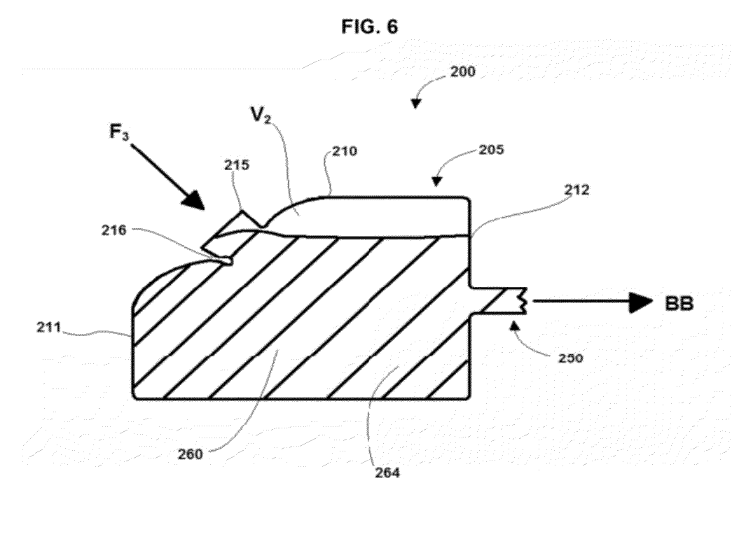

Although the medicament container 100 is shown and described as including an actuation portion 114 that, when moved from its first configuration to its second configuration, changes the rate of deformation of a portion of the medicament container, in other embodiments, a medicament container can be configured such that deformation of the medicament container can be propagated from a predetermined location of the container. Similarly stated, although the medicament container 100 is shown and described as including a wall having a temporally changing rate of deformation, in other embodiments, a medicament container can include a wall having a spatially variable rate of deformation (and/or a spatial variation in the stiffness of the wall). In this manner, the deformation of the wall can be propagated from a desired location and/or in a desired direction. For example, FIGS. 5 and 6 are schematic illustrations of a medicament container 200 according to an embodiment in a first configuration and a second configuration, respectively. The medicament container 200 includes a container portion 205 and a dispensing portion 250. The container portion 205 includes a wall 210 that defines, at least in part, a medicament reservoir 260 within which a medicament 264 can be disposed. The medicament 264 can be any suitable medicament, such as for example, a parasiticidal formula to be topically applied to an animal. In some embodiments, for example, the medicament 264 can be formulated to contain fipronil or a veterinarily acceptable derivative thereof. In some embodiments, the medicament 264 can be any of the formulations disclosed in U.S. Patent Publication No. 2011/0060023, entitled “Parasiticidal Formulation,” filed Mar. 18, 2010, which is incorporated herein by reference in its entirety.

The dispensing portion 250 can define an opening 222 (see e.g., FIG. 6) and is configured to place the medicament reservoir 260 in fluid communication with a volume outside of the medicament container 200. In this manner, the medicament 264 can be conveyed and/or dispensed from the medicament reservoir 260 via the dispensing portion 250, as described in more detail herein. The opening 222 can be defined by any suitable mechanism, such as, for example, by puncturing a portion of the dispensing portion 250, by removing a portion of the dispensing portion 250, by removing a cap, plug or other structure from the dispensing portion 250, or the like.

The wall 210 has a first end portion 211 and a second end portion 212, and includes a protrusion 215. As described herein, the wall 210 can be deformed and/or displaced (see FIG. 6) to reduce the volume of the medicament reservoir 260. More particularly, the wall 210 can be deformed when a force F₃ is applied to the protrusion 215 of the wall 210, as shown in FIG. 6. In this manner, the medicament 264 can be conveyed from the medicament reservoir 260 in response to the change in the volume of the medicament reservoir 260. Similarly stated, when the wall 210 is deformed and/or displaced, at least a portion of the force F₃ applied to the protrusion 215 acts upon the medicament 264 thereby causing the medicament to flow out of the medicament reservoir 260 via the dispensing portion 250.

The wall 210 and/or the protrusion 215 defines, at least in part, a stress concentration riser 216 configured to propagate the deformation of the wall 210 from a predetermined location of the wall 210. The stress concentration riser 216 can be any feature and/or mechanism that will promote deformation of the wall 210 in a predetermined location when the force F₃ is applied to the protrusion 215. Similarly stated, the stress concentration riser 216 can be any feature and/or mechanism that results in a spatial variation in the stiffness of the wall 210. In this manner, the first end portion 211 of the wall 210, which contains the stress concentration riser 216 has a lower stiffness (i.e., is less resistant to deformation and/or displacement when the force F₃ is applied) than the second end portion 212 of the wall 210. The stress concentration riser can include, for example, a portion of the wall 210 having a discontinuous shape, perforations defined by the wall 210 and/or the protrusion 215, an area of the wall 210 and/or the protrusion 215 having a reduced thickness (i.e., having a thickness that is less than a thickness of other portions of the wall 210) or the like. Thus, when the medicament container 200 is actuated, the first end portion 211 of the wall 210 will begin to deform before the second end portion 212 of the wall 210 begins to deform. Similarly stated, when the force F₃ is applied to the protrusion 215, the wall 210 will deform in a predetermined direction (i.e., from the first end portion 211 towards the second end portion 212, which is towards the dispensing portion 250). This arrangement results in consistent and/or complete delivery of the medicament 264.

As shown in FIGS. 5 and 6, the medicament container 200 can be moved between a first (or storage) configuration (see FIG. 5) and a second (or actuation) configuration (see FIG. 6). When the medicament container 200 is in the first configuration, the medicament reservoir 260 is fluidically isolated from the volume outside of the medicament container 200. Similarly stated, the container portion 205 defines a substantially hermetic and/or fluid-tight seal to prevent leakage of the medicament 264 from the medicament reservoir 260. When the medicament container 200 is in the first configuration, the medicament reservoir 260 has a volume V₁.

The medicament container 200 is moved from its first configuration (FIG. 5) to its second configuration (FIG. 6) when the opening 222 is defined by the dispensing portion 250, and the force F₃ is applied to the protrusion 215. As described above, the force F₃ causes the wall 210 to deform, beginning at a predetermined location of the wall 210, as described above. The deformation, displacement and/or deflection of the portion of the wall 210 when the medicament container 200 is moved to its second configuration reduces the volume of the medicament reservoir 260 to the volume V₂, as shown in FIG. 6. The reduction in the volume of the medicament reservoir 260 results in at least a portion of the medicament 264 being conveyed and/or dispensed from the medicament reservoir 260 via the dispensing portion 250, as shown by the arrow BB in FIG. 6.

The deformation, displacement and/or deflection of the portion of the wall 210 when the medicament container 200 is moved to its second configuration can be at any suitable rate. In some embodiments, the deformation, displacement and/or deflection of the portion of the wall 210 can occur at a substantially constant rate through the actuation event. In other embodiments, the rate of deformation can vary temporally, as described above with reference to the medicament container 100.

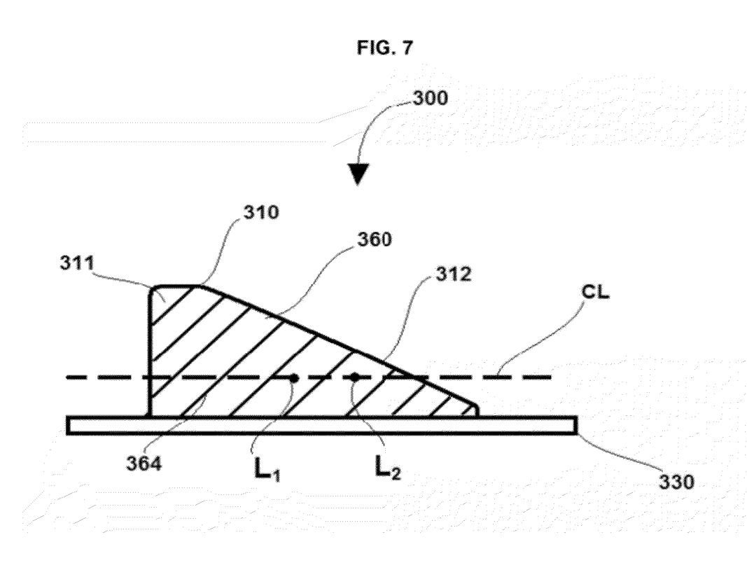

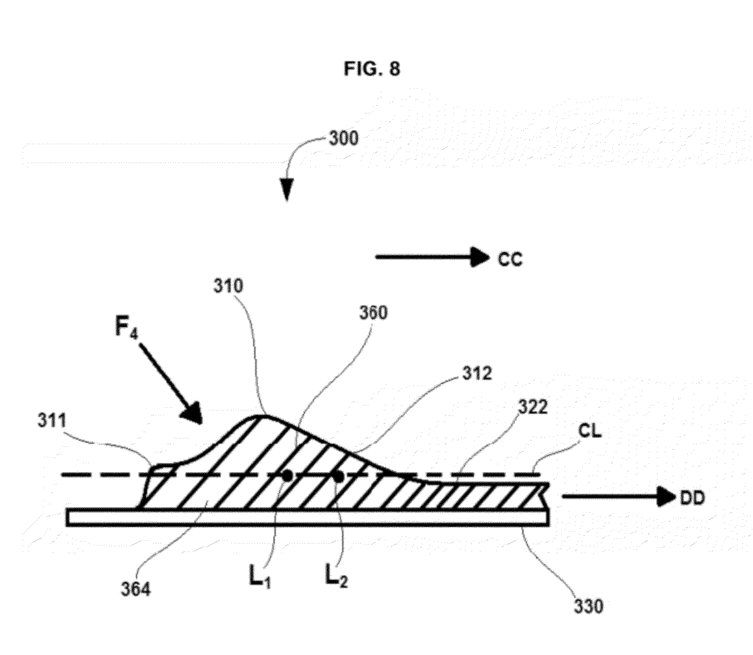

FIGS. 7 and 8 are schematic illustrations of a medicament container 300 according to an embodiment in a first configuration and a second configuration, respectively. The medicament container 300 includes a first layer 330 and a second layer 310 coupled to the first layer 330. The second layer 310 defines a medicament reservoir 360 within which a medicament 364 can be disposed. The medicament 364 can be any suitable medicament, such as for example, a parasiticidal formula to be topically applied to an animal. In some embodiments, for example, the medicament 364 can be formulated to contain fipronil or a veterinarily acceptable derivative thereof. In some embodiments, the medicament 364 can be any of the formulations disclosed in U.S. Patent Publication No. 2011/0060023, entitled “Parasiticidal Formulation,” filed Mar. 18, 2010, which is incorporated herein by reference in its entirety.

Although the medicament reservoir 360 is shown as being substantially fully filled with the medicament 364, in other embodiments, the medicament reservoir 360 can be partially filled with the medicament 364. Similarly stated, in some embodiments, the volume of the medicament 364 when the medicament container 300 is in the first (or storage) configuration, as shown in FIG. 7, is less than the volume of the medicament reservoir 360 defined by the second layer 310.

The second layer 310 can define an opening 322 (see e.g., FIG. 8) through which the medicament reservoir 360 can be placed in fluid communication with a volume outside of the medicament container 300. In this manner, the medicament 364 can be conveyed and/or dispensed from the medicament reservoir 360 via the opening 322, as described in more detail herein. The opening 322 can be defined by any suitable mechanism, such as, for example, by puncturing a portion of the second layer 310, by removing a portion of the second layer 310, by removing a cap, plug or other structure from the second layer 310, or the like.

The second layer 310 has a first end portion 311 and a second end portion 312, and defines a center line CL. As described herein, the second layer 310 can be deformed and/or displaced (see FIG. 8) to reduce the volume of the medicament reservoir 360. More particularly, the second layer 310 can be deformed when a force F₄ is applied to an actuation portion 314 of the second layer 310, as shown in FIG. 8. In this manner, the medicament 364 can be conveyed from the medicament reservoir 360 in response to the change in the volume of the medicament reservoir 360. Similarly stated, when the second layer 310 is deformed and/or displaced, at least a portion of the force F₄ applied to the actuation portion 314 acts upon the medicament 364 thereby causing the medicament to flow out of the medicament reservoir 360 via the opening 322, as shown by the arrow DD in FIG. 8.

As shown in FIG. 7, the second end portion 312 of the second layer 310 is tapered along the center line CL. Similarly stated, the second end portion 312 of the second layer 310 is configured such that a cross-sectional area (not shown in FIGS. 7 and 8) of the medicament reservoir 360 taken at a first location L₁ along the center line CL is greater than a cross-sectional area of the medicament reservoir 360 taken at a second location L₂ along the center line CL. Thus, the cross-sectional area (or flow path) of the medicament reservoir 360 decreases in the direction indicated by the arrow CC, which is towards the opening 322. Although the second end portion 312 of the second layer 310 is shown as being tapered in a single dimension (i.e., height dimension as depicted in FIG. 7), in other embodiments, the second end portion 312 can be tapered in two dimensions (e.g., a height dimension and a width dimension).

The actuation portion 314 is configured to propagate the deformation of the second layer 310 from the first location L₁ towards the second location L₂. Similarly stated, the actuation portion 314 is configured to propagate the deformation of the second layer 310 in the direction shown by the arrow CC in FIG. 8. In this manner, when the medicament container 300 is actuated, the direction of deformation will cause the medicament 364 to be conveyed towards the opening 322. This arrangement results in consistent and/or complete delivery of the medicament 364.

The actuation portion 314 can include any suitable mechanism and/or feature to propagate the deformation of the second layer 310 in the direction shown by the arrow CC in FIG. 8. For example, in some embodiments the actuation portion 314 can include one or more stress concentration risers of the types shown and described herein. In other embodiments, the actuation portion 314 can be constructed from a different material than the remainder of the second layer 310, thereby resulting in a spatial variation in the stiffness of the second layer 310.

As shown in FIGS. 7 and 8, the medicament container 300 can be moved between a first (or storage) configuration (see FIG. 7) and a second (or actuation) configuration (see FIG. 8). When the medicament container 300 is in the first configuration, the medicament reservoir 360 is fluidically isolated from the volume outside of the medicament container 300. Similarly stated, the first layer 330 and the second layer 310 collectively define a substantially hermetic and/or fluid-tight seal to prevent leakage of the medicament 364 from the medicament reservoir 360. When the medicament container 300 is in the first configuration, the medicament reservoir 360 has a volume V₁.

The medicament container 300 is moved from its first configuration (FIG. 7) to its second configuration (FIG. 8) when the opening 322 is defined in the second end portion 312 of the second layer 310, and the force F₄ is applied to the actuation portion 314. As described above, the force F₄ causes the second layer 310 to deform in the direction indicated by the arrow CC in FIG. 8, as described above. The deformation, displacement and/or deflection of the portion of the second layer 310 when the medicament container 300 is moved to its second configuration reduces the volume of the medicament reservoir 360 to the volume V₂, as shown in FIG. 8. The reduction in the volume of the medicament reservoir 360 results in at least a portion of the medicament 364 being conveyed and/or dispensed from the medicament reservoir 360 via the opening 322, as shown by the arrow DD in FIG. 8.

The deformation, displacement and/or deflection of the portion of the second layer 310 when the medicament container 300 is moved to its second configuration can be at any suitable rate. In some embodiments, the deformation, displacement and/or deflection of the portion of the second layer 310 can occur at a substantially constant rate through the actuation event. In other embodiments, the rate of deformation can vary temporally, as described above with reference to the medicament container 100.

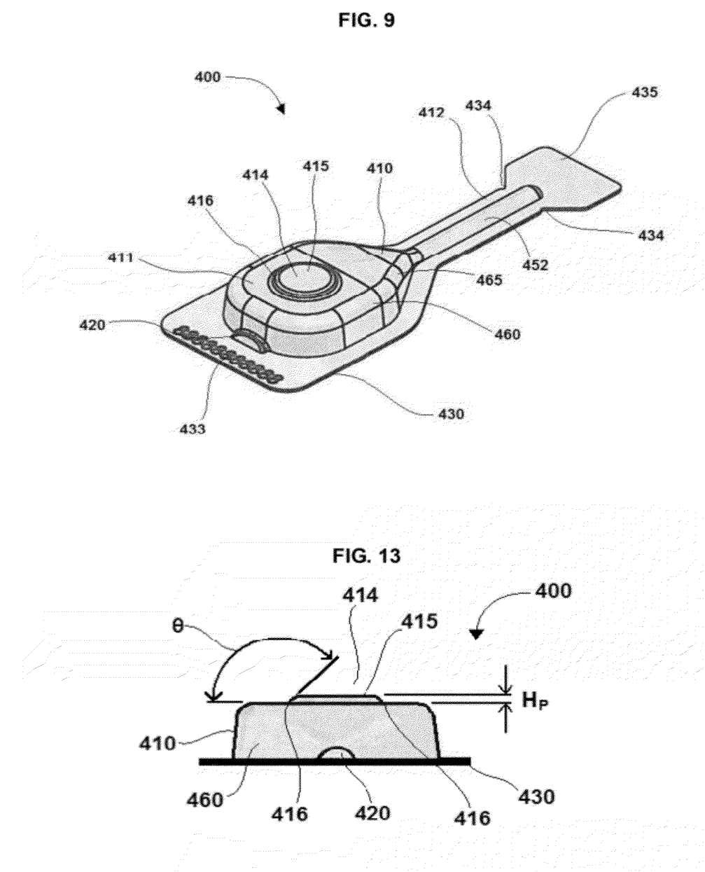

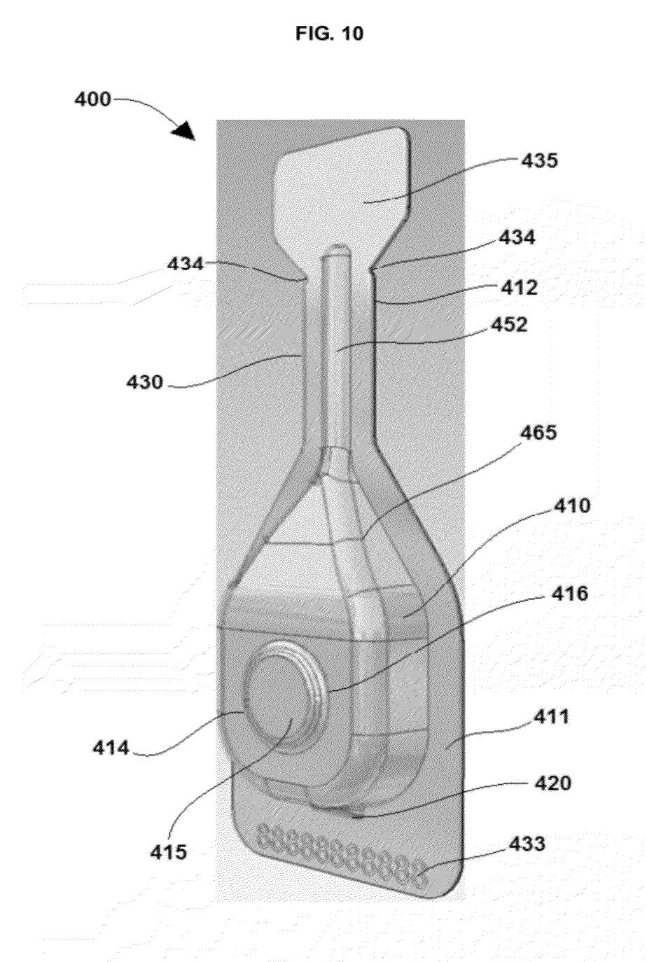

FIGS. 9-15 show various views of a medicament container 400 according to an embodiment. The medicament container 400 has a proximal end portion 411 and a distal end portion 412, and includes a first layer 430 and a second layer 410 coupled to the first layer 430. The second layer 410 defines a medicament reservoir 460 at the proximal end portion 411, and a delivery lumen 452 in fluid communication with the medicament reservoir 460 at the distal end portion 412. The first layer 430 defines two stress concentration risers 434 and includes a tip 435 at the distal end portion 412 of the medicament container 400. The stress concentration risers 434 are configured to propagate deformation and/or disruption of the first layer 430 such that when a force is applied to the tip 435, an opening (not shown) in fluid communication with the delivery lumen 452 is defined. In this manner, the medicament container 400 can be “opened” when the user twists or otherwise exerts a force on the tip 435. A medicament 464 can then be conveyed and/or dispensed from the medicament reservoir 460 via the delivery lumen 452. In this manner, the distal end portion 412 can function as a delivery tube or pipette to deliver the medicament 464 to a desired location, such as, for example, topically to the skin of an animal.

Although the stress concentration risers 434 are shown as being tapered notches defined by the first layer 430 of the medicament container 400, in other embodiments, the first layer 430 can define any suitable stress concentration riser to propagate deformation and/or disruption of the first layer 430 in a desired direction. For example, in some embodiments, the first layer 430 and/or the second layer 410 can define a series of perforations that form a boundary of the opening to be defined when the tip 435 is twisted.

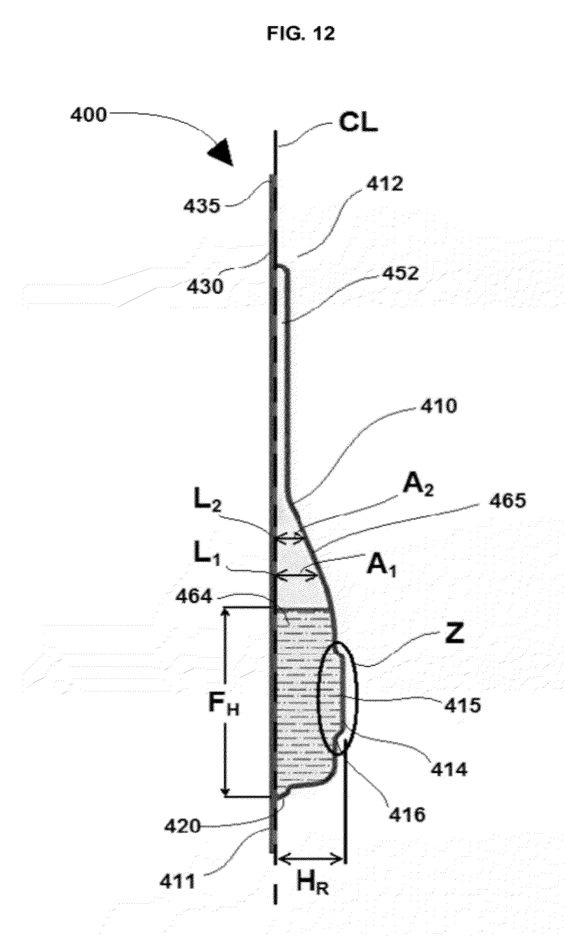

The medicament reservoir 460 can contain any suitable medicament 464 (see, e.g., FIG. 12), such as for example, a parasiticidal formula to be topically applied to an animal. In some embodiments, for example, the medicament 464 can be formulated to contain fipronil or a veterinarily acceptable derivative thereof. In some embodiments, the medicament 464 can be any of the formulations disclosed in U.S. Patent Publication No. 2011/0060023, entitled “Parasiticidal Formulation,” filed Mar. 18, 2010, which is incorporated herein by reference in its entirety.

As shown in FIG. 12, the medicament reservoir 460 is partially filled with the medicament 464. More particularly, when the medicament container 400 is placed in a substantially vertical position, the medicament 464 contained therein has a fill height F_{H} that is less than the total height of the medicament reservoir. Thus, the dose of medicament 464 contained within the medicament reservoir 460 can be adjusted and/or controlled by adjusting and/or controlling the fill height F_{H} of the medicament 464. The dose of medicament 464 contained within the medicament reservoir 464 can be any suitable dose volume, such as, for example, a nominal dose volume of 0.5 ml, 0.6 ml, 1.34 ml, 2.7 ml or 4.2 ml. Although the medicament reservoir 460 is shown as being only partially filled with the medicament 464, in other embodiments, the medicament reservoir 460 can be substantially entirely filled with the medicament 464.

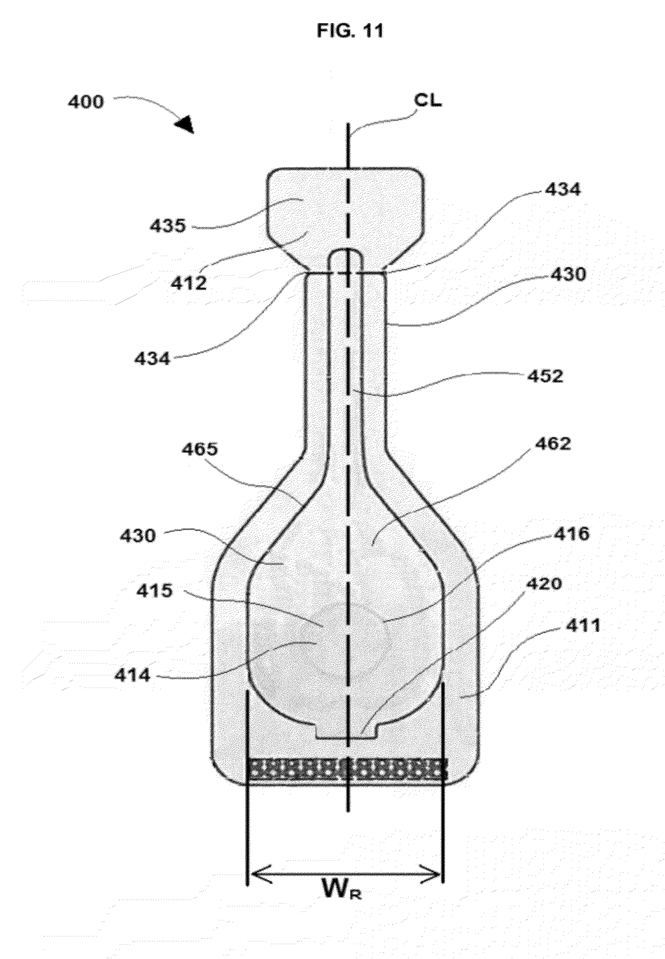

In addition to adjusting and/or controlling the volume of medicament 464 contained within the medicament container 460 by adjusting the fill height F_{H}, the volume of the medicament 464 is also a function of the height H_{R} (see e.g., FIG. 12) and the width W_{R} (see FIG. 11) of the medicament reservoir 460. As discussed in more detail below, the height H_{R} and width W_{R} can be any suitable height H_{R} and width W_{R} to produce the desired volume of the medicament reservoir 460. For example, in some embodiments, the nominal height H_{R} can be approximately 8 mm and the nominal width W_{R} can be approximately 26 mm. In such embodiments, when the fill height F_{H} is approximately 26 mm, the volume of the medicament 464 within the medicament reservoir 460 is approximately 4.2 ml.

The second layer 410 defines a fill port 420 through which the medicament 464 can be conveyed into the medicament reservoir 460 during the assembly and fill process. The proximal end portion 411 includes a seal 433 that fluidically isolates (or closes) the fill port 420 after filling is complete. The seal 433 can be formed by any suitable mechanism. In some embodiments, a portion of the first layer 430 can be welded and/or thermally bonded to a portion of the second layer 430 to define the seal 433. In other embodiments, a portion of the first layer 430 can be bonded to a portion of the second layer 430 by an adhesive to define the seal 433.

As shown in FIGS. 11 and 12, the second layer 410 includes a tapered portion 465 that is tapered along a longitudinal center line CL of the medicament container 400. Similarly stated, the tapered portion 465 of the second layer 410 is configured such that a cross-sectional area A₁ of the medicament reservoir 460 taken at a first location L₁ along the center line CL is greater than a cross-sectional area A₂ of the medicament reservoir 460 taken at a second location L₂ along the center line CL. Thus, the cross-sectional area (or flow path) of the medicament reservoir 460 decreases in the direction towards the delivery lumen 452. Although the tapered portion 465 is shown as being tapered in two dimensions (i.e., a height dimension and a width dimension), in other embodiments, the tapered portion 465 need only be tapered in one direction (e.g., either a height or a width).

The second layer 410 includes an actuation portion 414 that has a protrusion 415 extending from the medicament reservoir 460. The actuation portion 414 and/or the protrusion 415 define a series of stress concentration risers 416. As described in more detail herein, when a force is applied to the actuation portion 414 and/or the protrusion 415 (e.g., by being depressed by a user), at least a portion of the second layer 410 can deform, thereby causing at least a portion of the medicament 464 to be conveyed and/or delivered from the medicament reservoir 460 via the delivery lumen 453.

As shown in FIG. 13, the protrusion 415 extends from the medicament reservoir 460 by a distance H_{P}. The distance H_{P} can be any suitable distance such that the protrusion 415 and/or the actuation portion 414 sufficiently define the stress concentration risers 416. For example, in some embodiments, the distance H_{P} can be approximately 1 mm. In other embodiments, the distance H_{P} can be approximately 2 mm, approximately 3 mm or approximately 5 mm. Although the protrusion 415 is shown as having a substantially circular shape, in other embodiments, the protrusion 415 can have any suitable shape (e.g., oval, oblong, triangular, rectangular or the like). The actuation portion 414 and/or the protrusion 415 define the series of stress concentration risers 416. More particularly, the stress concentration risers 416 are the annular boundaries at which the height of the second layer 430 is changed to form the protrusion 415. Thus, the stress concentration risers 416 substantially circumscribe the protrusion 415.

As shown in FIG. 13, the portion of the second layer 410 that defines the protrusion 415 and/or the stress concentration risers 416 defines an angle Θ. Although shown as being an obtuse angle, the angle Θ can have any suitable value. In some embodiments, for example, the angle Θ can be acute (less than 90 degrees), which produces an undercut between the protrusion 415 and the remainder of the actuation portion 414. In other embodiments, the angle Θ can be approximately 90 degrees.

The stress concentration risers 416 are configured to propagate the deformation of the second layer 410 from a predetermined location of the second layer 410. Similarly stated, the stress concentration risers 416 produce a spatial variation in the stiffness of the second layer 410. More particularly, the actuation portion 414 has a lower stiffness (i.e., is less resistant to deformation and/or displacement when a force is applied) than other portions of the second layer 410. Thus, when the medicament container 400 is actuated, the proximal end portion 411 of the wall 410 will begin to deform before the distal end portion 412 of the wall 410 begins to deform. Similarly stated, when the actuation force is applied to the protrusion 415 and/or the actuation portion 414, the second layer 410 will deform in a predetermined direction (i.e., proximal to distal). In this manner, when the medicament container 400 is actuated, the direction of deformation will cause the medicament 464 to be conveyed towards the tapered portion of the second layer 410 and/or the delivery lumen 452. This arrangement results in consistent and/or complete delivery of the medicament 464.

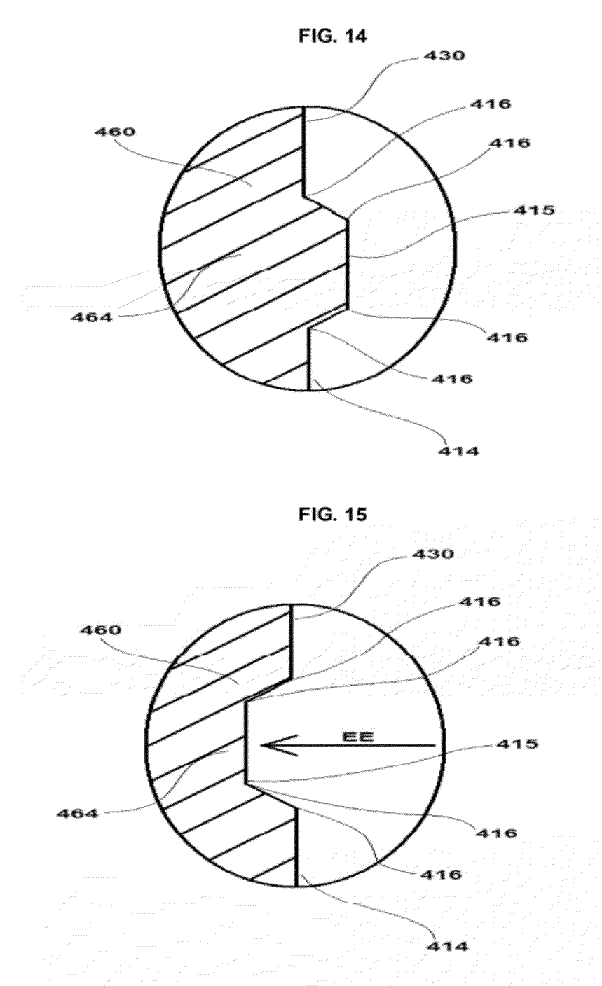

As shown in FIGS. 14 and 15, when the actuation force is applied to the actuation portion 414, the actuation portion 414 moves substantially suddenly and/or discontinuously from a first configuration to a second configuration. More particularly, FIGS. 14 and 15 show the portion of the second layer identified as portion Z in FIG. 12 in a first configuration and a second configuration, respectively. When the actuation force applied to the actuation portion 414 exceeds a threshold, the portion of the second layer 410 surrounding the protrusion 415 substantially suddenly and/or discontinuously changes (or “buckles”) as shown by the arrow EE in FIG. 15. In this manner, the configuration of the actuation portion 414 produces a temporally varying rate of deformation of the second layer 410, in a manner similar to that described above with reference to the medicament container 100. The varying rate of deformation results in consistent delivery of a desired dose of the medicament 464 during actuation of the medicament container 400, as described above.

The first layer 430 and the second layer 410 can have any suitable thickness. For example, the first layer 430 and/or the second layer 410 can have a thickness of less than 1 mm, less than 500 microns, less than 200 microns or less than 100 microns. In some embodiments, the first layer 430 and the second layer 410 can have substantially the same thickness. In other embodiments, a thickness of the first layer 430 can be greater than a thickness of the second layer 410. In such embodiments, the differences in thickness produce a first layer 430 that is stiffer (or more resistant to deformation) than a second layer 410. Accordingly, during actuation of the medicament container 400, the second layer 410, and in particular, the actuation portion 414 of the second layer 410, will deform before and/or faster than the first layer 430. In this manner, the deformation of the medicament container 400 can propagate in a desired direction (e.g., proximal to distal) and/or at a desired rate to facilitate consistent delivery of the medicament 464, as described herein.

Moreover, the first layer 430 and/or the second layer 410 can have a spatial variation in its thickness. For example, the portion of the second layer 410 that defines the delivery lumen 452 can have a greater thickness than the actuation portion 414 of the second layer 410. In this manner, the distal end portion 412 (i.e., the portion through which the medicament 464 is delivered) can have a higher stiffness than the actuation portion 414. This arrangement can reduce the likelihood that the delivery lumen 452 will collapse and/or be obstructed by inadvertent deformation of the second layer 410 during use and/or handling of the medicament container 400.

The first layer 430 and/or the second layer 410, as well as any of the other layers, walls and structures included within any of the medicament containers described herein can be constructed from any suitable material. Such materials can be selected to minimize interaction with the medicament 464. For example, in some embodiments, the first layer 430 and/or the second layer 410 can be constructed from a substantially inert and/or flexible polymer. More particularly, in some embodiments, the first layer 430 and/or the second layer 410 can be constructed from flexible polymers, such as polyesters, polyamides, polypropylenes and/or polyolefins. In other embodiments, the first layer 430 can be constructed from a polymer have a first hardness that is greater than the hardness of a material from which the second layer 410 is constructed.

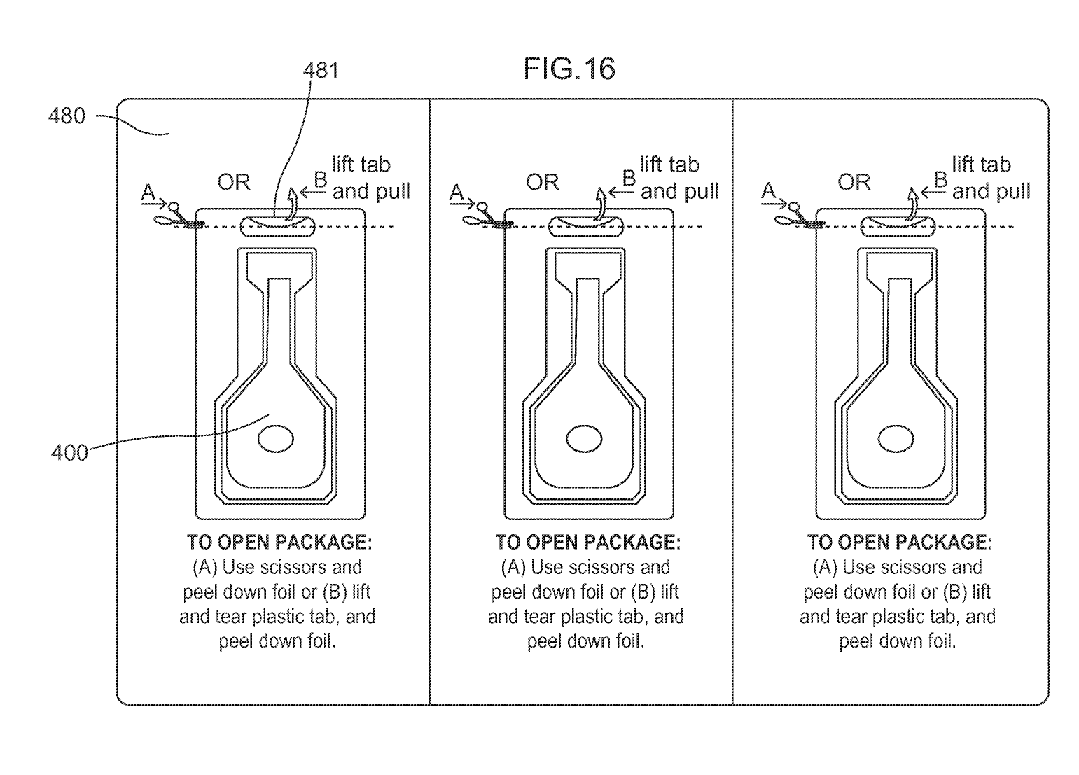

FIG. 16 shows a package 480 within which one or more medicament containers 400 can be disposed for storage and handling. Although the package 480 is shown as including three medicament containers 400, in other embodiments, a package can be configured to contain any number of medicament containers. The package is a constructed from a flexible foil and includes an actuator tab 481 and instructions to facilitate opening the foil.

To use the medicament container 460 to deliver the medicament 464, the user first opens the package 480 and removes the medicament container 400 disposed therein. The user then applies a force (e.g., a twisting force) to the tip 435 at the distal end portion 412 of the medicament container to produce an opening (not shown) in fluid communication with the delivery lumen 452. In some embodiments, this operation will result in removal of the tip 435 from the medicament container 400. In other embodiments, however, a portion of the tip 435 can remain coupled to the distal end portion 412 of the medicament container 400.

The distal end portion 412 is then placed adjacent the target location (e.g., the skin of an animal) and the medicament container 400 is actuated by applying a force to the actuation portion 414 of the second layer 410. Said another way, after the distal end portion 412 is positioned in the desired location, the user squeezes the actuation portion 414 to convey a portion of the medicament 464 from the medicament reservoir 460 to the target location via the delivery lumen 452. More particularly, when the medicament container 410 is actuated, at least a portion of the second layer 410 is deformed and/or displaced, in the direction and manner as described above. The deformation of the second layer 410 reduces the volume of the medicament reservoir 460. In this manner, the medicament 464 can be conveyed from the medicament reservoir 460 in response to the change in the volume of the medicament reservoir 460. Similarly stated, when the second layer 410 is deformed and/or displaced, at least a portion of the force applied to the actuation portion 414 acts upon the medicament 464 thereby causing the medicament to flow out of the medicament reservoir 460 via the delivery lumen 452.

As discussed above, the height H_{R} and width W_{R} can be any suitable height H_{R} and width W_{R} to produce the desired volume of the medicament reservoir 460. For example, in some embodiments, the nominal height H_{R} can be within the range of approximately 2 mm to approximately 8 mm and the nominal width W_{R} can be within the range of approximately 20 mm to approximately 28 mm. Moreover, the nominal fill height F_{H} can be within the range of approximately 18 mm to approximately 26 mm.

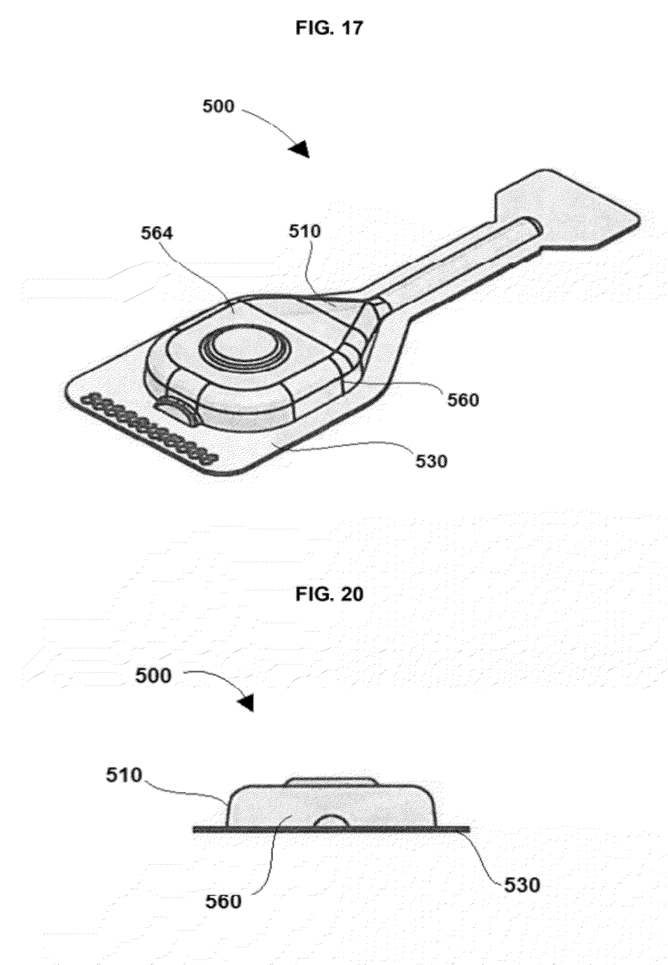

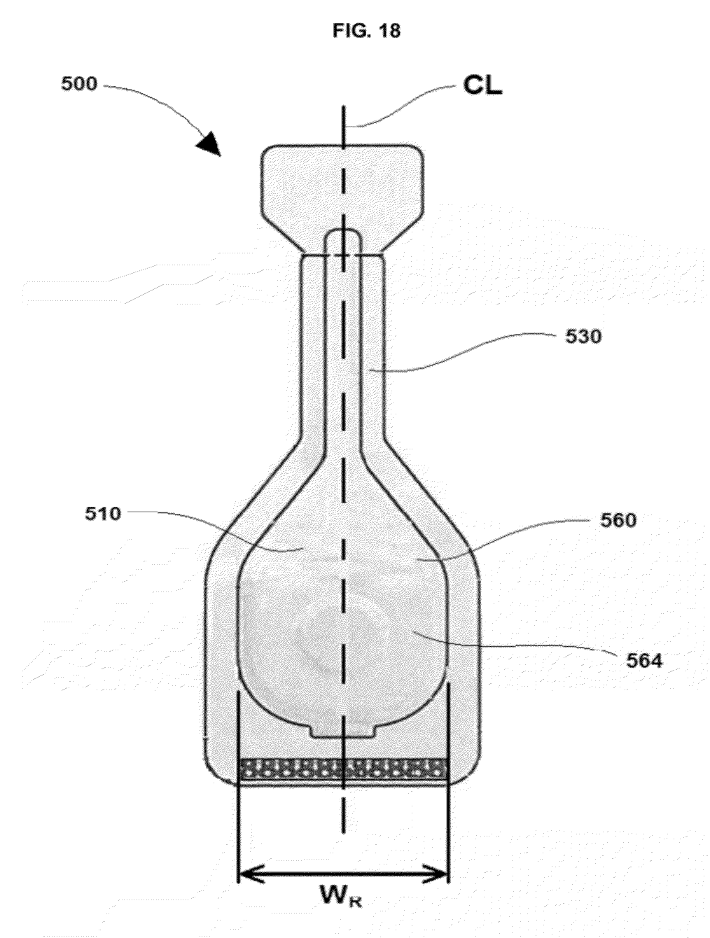

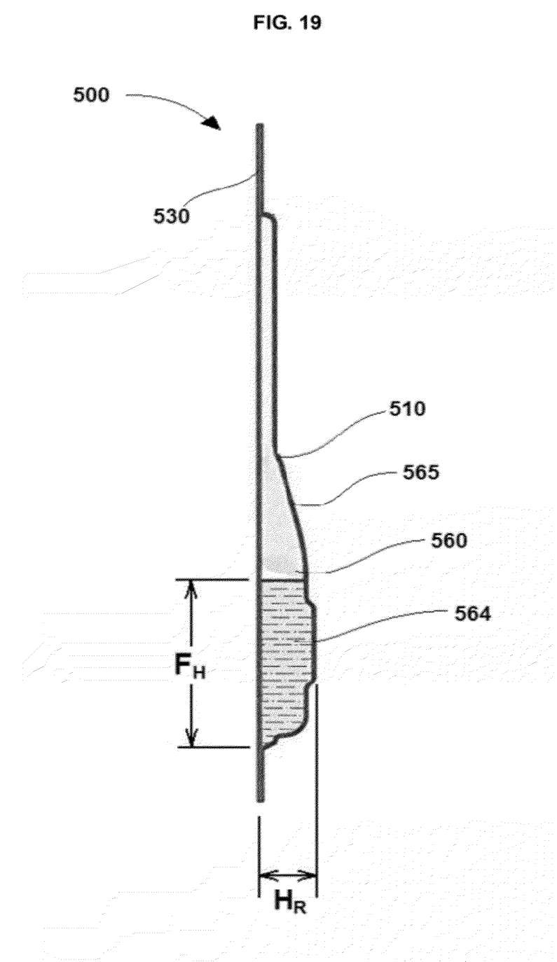

FIGS. 17-20 show various views of a medicament container 500 according to an embodiment. Similar to the medicament container 400, the medicament container 500 includes a first layer 530 and a second layer 510, and defines a medicament reservoir 560 that contains a medicament 564. The structure and function of the medicament container 500 are similar to the structure and function of the medicament container 400, and are therefore not described in detail herein. The medicament container 500 differs from the medicament container 400, however, in that the nominal height H_{R} (see, e.g., FIG. 19) of the medicament reservoir 560 is less than the nominal height H_{R} (see, e.g., FIG. 12) of the medicament reservoir 460. In some embodiments, the nominal height H_{R} of the medicament reservoir 560 can be approximately 6 mm and the nominal width W_{R} of the medicament reservoir 560 can be approximately 26 mm. In such embodiments, when the fill height F_{H} is approximately 23 mm, the volume of the medicament 564 within the medicament reservoir 560 is approximately 2.7 ml.

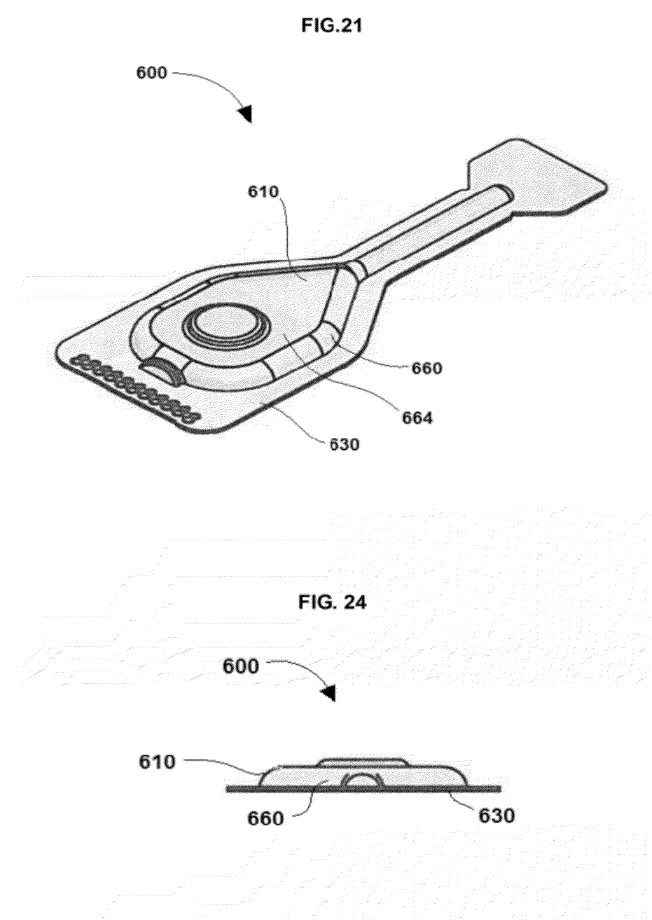

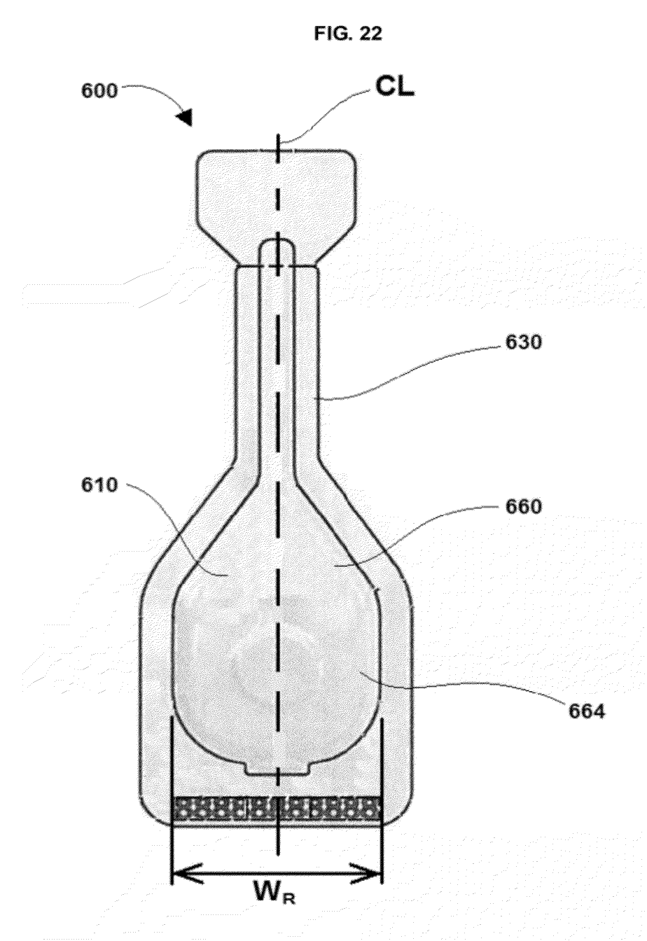

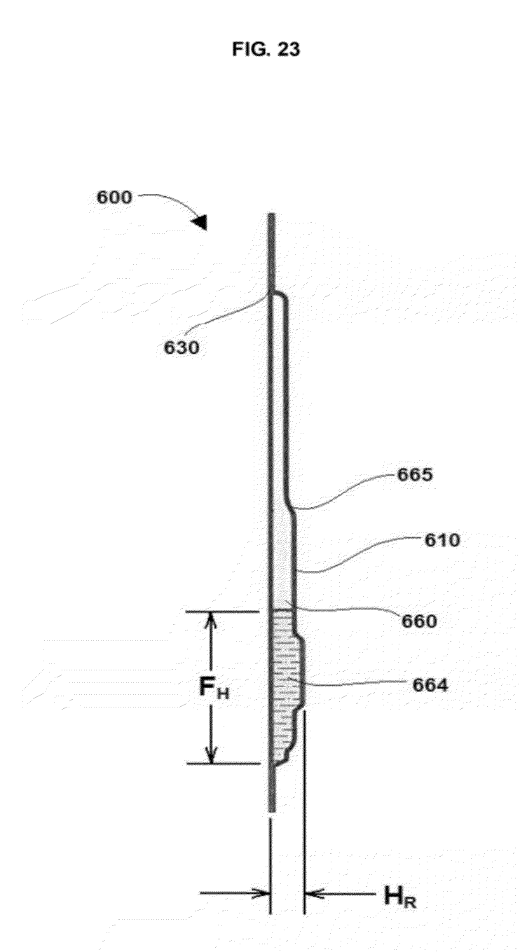

FIGS. 21-24 show various views of a medicament container 600 according to an embodiment. Similar to the medicament container 400, the medicament container 600 includes a first layer 630 and a second layer 610, and defines a medicament reservoir 660 that contains a medicament 664. The structure and function of the medicament container 600 are similar to the structure and function of the medicament container 400, and are therefore not described in detail herein. The medicament container 600 differs from the medicament container 400, however, in that the nominal height H_{R} (see, e.g., FIG. 23) of the medicament reservoir 660 is less than the nominal height H_{R} (see, e.g., FIG. 12) of the medicament reservoir 460. In some embodiments, the nominal height H_{R} of the medicament reservoir 660 can be approximately 3 mm and the nominal width W_{R} of the medicament reservoir 660 can be approximately 26 mm. In such embodiments, when the fill height F_{H} is approximately 23.5 mm, the volume of the medicament 664 within the medicament reservoir 660 is approximately 1.34 ml.

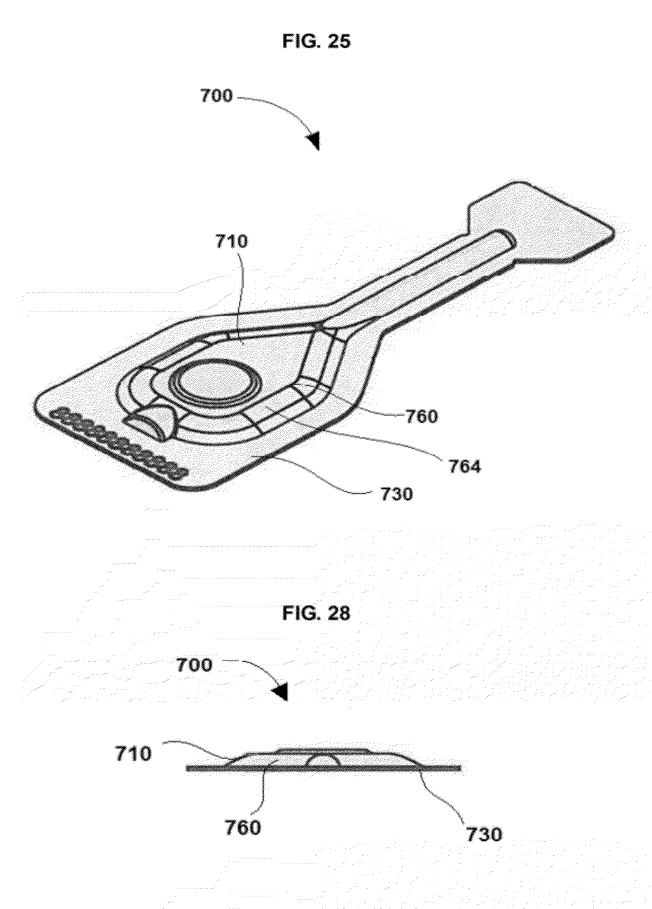

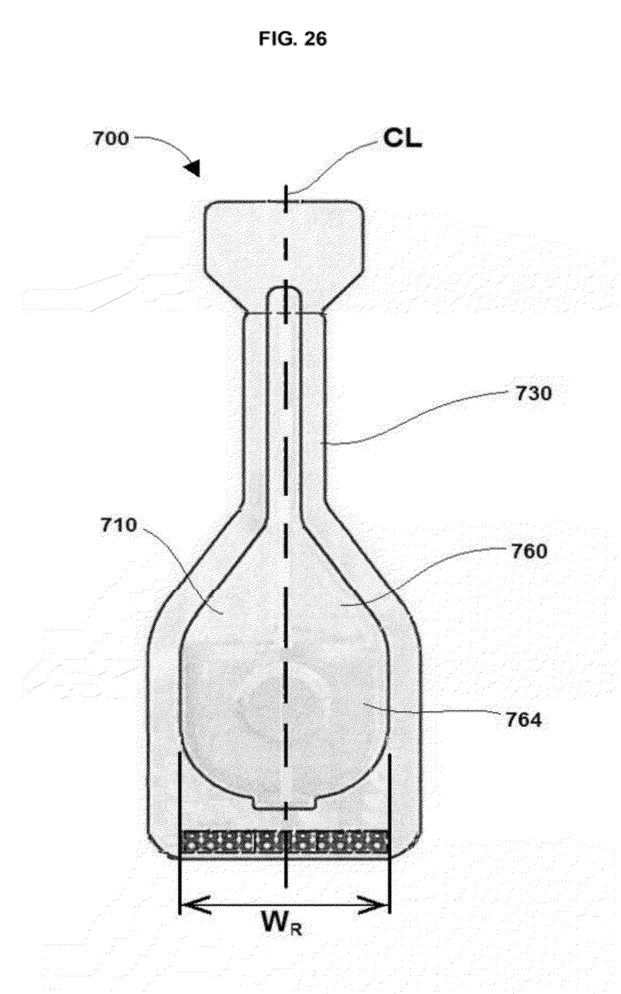

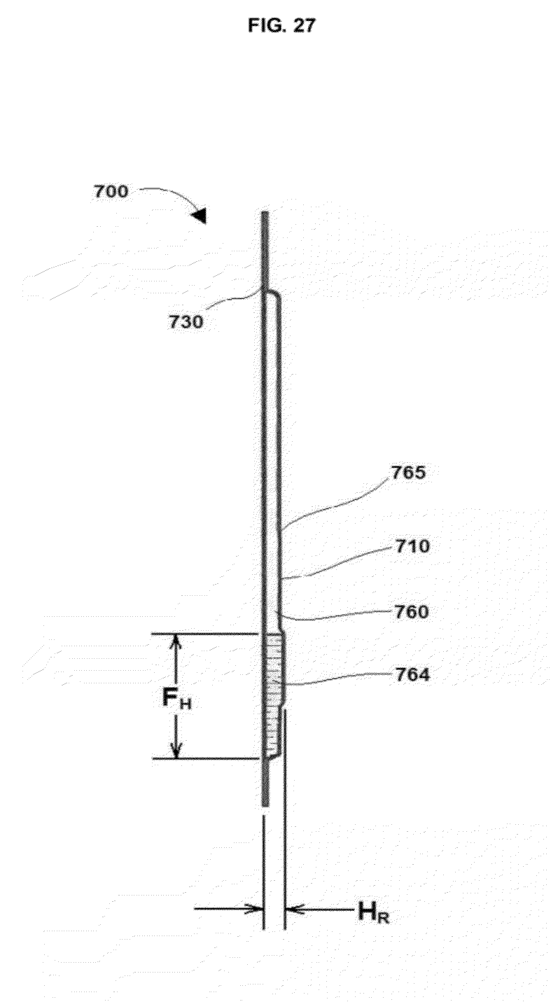

FIGS. 25-28 show various views of a medicament container 700 according to an embodiment. Similar to the medicament container 400, the medicament container 700 includes a first layer 730 and a second layer 710, and defines a medicament reservoir 760 that contains a medicament 764. The structure and function of the medicament container 700 are similar to the structure and function of the medicament container 400, and are therefore not described in detail herein. The medicament container 700 differs from the medicament container 400, however, in that the nominal height H_{R} (see, e.g., FIG. 27) and the nominal width W_{R} (see, e.g. FIG. 26) of the medicament reservoir 760 are less than the nominal height H_{R} (see, e.g., FIG. 12) the nominal width W_{R} (see, e.g. FIG. 11) of the medicament reservoir 460. In some embodiments, the nominal height H_{R} of the medicament reservoir 760 can be approximately 2.1 mm and the nominal width W_{R} of the medicament reservoir 760 can be approximately 25 mm. In such embodiments, when the fill height F_{H} is approximately 19.5 mm, the volume of the medicament 764 within the medicament reservoir 760 is approximately 0.6 ml.

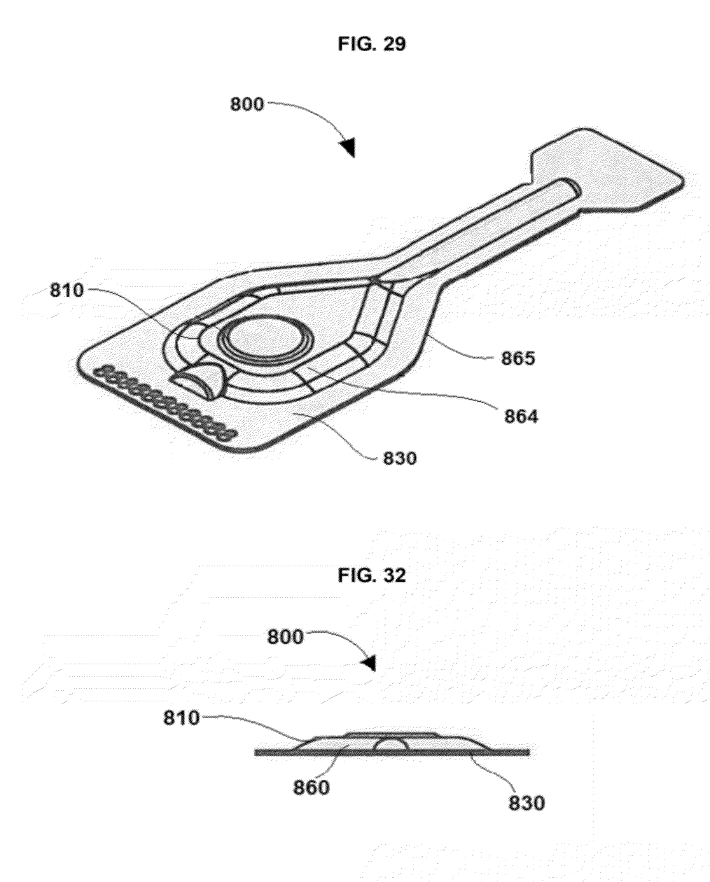

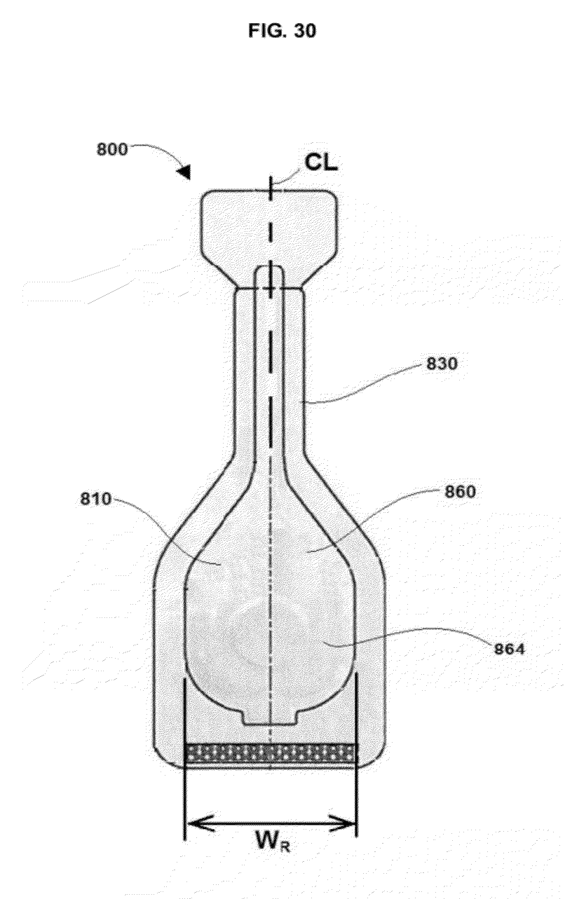

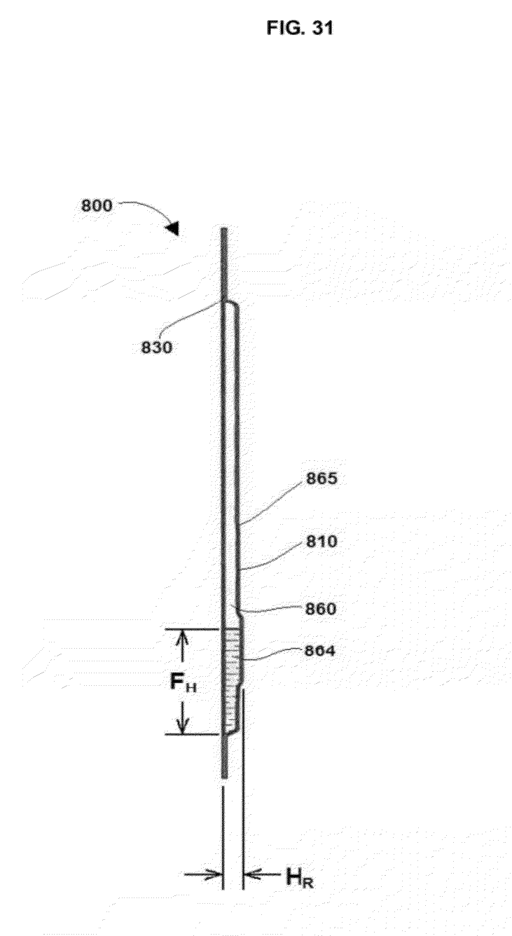

FIGS. 29-32 show various views of a medicament container 800 according to an embodiment. Similar to the medicament container 400, the medicament container 800 includes a first layer 830 and a second layer 810, and defines a medicament reservoir 860 that contains a medicament 864. The structure and function of the medicament container 800 are similar to the structure and function of the medicament container 400, and are therefore not described in detail herein. The medicament container 800 differs from the medicament container 400, however, in that the nominal height H_{R} (see, e.g., FIG. 31) and the nominal width W_{R} (see, e.g. FIG. 30) of the medicament reservoir 860 are less than the nominal height H_{R} (see, e.g., FIG. 12) the nominal width W_{R} (see, e.g. FIG. 11) of the medicament reservoir 460. In some embodiments, the nominal height H_{R} of the medicament reservoir 860 can be approximately 2.1 mm and the nominal width W_{R} of the medicament reservoir 860 can be approximately 25 mm. In such embodiments, when the fill height F_{H} is approximately 18 mm, the volume of the medicament 864 within the medicament reservoir 860 is approximately 0.5 ml.

While various embodiments have been described above, it should be understood that they have been presented by way of example only, and not limitation. Where methods and/or schematics described above indicate certain events and/or flow patterns occurring in certain order, the ordering of certain events and/or flow patterns may be modified. Additionally certain events may be performed concurrently in parallel processes when possible, as well as performed sequentially. While the embodiments have been particularly shown and described, it will be understood that various changes in form and details may be made.

Although the substrate 130 and the wall 110 are shown and described as being separate structures that are coupled together, in other embodiments, the substrate 130 and the wall can be monolithically formed. Similarly, although the medicament container 400 is shown and described as including a first layer 430 and a second layer 410, in other embodiments, the medicament container 400 can be monolithically constructed. Moreover, although the second layer 410 is shown and described as defining both the medicament reservoir 460 and the delivery lumen 452, in other embodiments, the delivery lumen 452 can be defined by a separate structure than the structure that defines the medicament reservoir 460.

Although the actuation portion 114 is shown as changing its size and/or shape when moved from its first configuration (see e.g., FIGS. 1 and 2) and its second configuration (see e.g., FIG. 3), in other embodiments an actuation portion can moved between a first configuration and a second configuration while maintaining a substantially constant size and/or shape. In some embodiments, an actuation portion 114 can change between a first configuration and a second configuration substantially suddenly and/or discontinuously, thereby producing a temporal change in a deformation rate of a container, while maintaining a substantially constant size and/or shape.

Although the stress concentration risers 416 are shown as being annular boundaries at which the height of the second layer 430 is changed to form the protrusion 415, in other embodiments, the stress concentrations risers can be any feature and/or mechanism that will promote deformation of the second layer 410 in a predetermined location when the force is applied to the protrusion 415. For example, in some embodiments, the stress concentration risers 416 can include, a portion of the second layer 410 having perforations, a reduced thickness (i.e., having a thickness that is less than a thickness of other portions of the second layer 410) or the like. In other embodiments, the actuation portion 414 can be constructed from a different material than the remainder of the second layer 410, thereby resulting in a spatial variation in the stiffness of the second layer 410.

Although various embodiments have been described as having particular features and/or combinations of components, other embodiments are possible having a combination of any features and/or components from any of embodiments where appropriate. For example, in some embodiments, the medicament container 100 that is depicted schematically in FIGS. 1-3 can include a distal end portion defining a delivery lumen similar to the distal end portion 412 of the medicament container 400 that defines the delivery lumen 452.

What is claimed is:

1. An apparatus, comprising:
a substrate; and
a wall coupled to the substrate, the substrate and the wall defining a medicament reservoir, at least a portion of the wall configured to be deformed to reduce a volume of the medicament reservoir when the wall is actuated, the wall including an actuation portion, the portion of the wall configured to deform at a first rate when the actuation portion is in a first configuration, the wall configured to deform at a second rate when the actuation portion is in a second configuration.

2. The apparatus of claim 1, wherein the wall is a first wall, the apparatus further comprising:
a second wall coupled to the substrate, the substrate and the second wall defining a lumen in fluid communication with the medicament reservoir,
a distal end portion of the substrate defining a stress concentration riser configured to propagate deformation of the substrate in a predetermined direction when a force is exerted on the distal end portion of the substrate.

3. The apparatus of claim 1, wherein the actuation portion includes a protrusion extending from the medicament reservoir.

4. The apparatus of claim 1, wherein the actuation portion includes a detent mechanism configured to resist deformation of the wall.

5. The apparatus of claim 1, wherein the actuation portion includes a stress concentration riser configured to propagate deformation of the wall from a predetermined location of the wall.

6. The apparatus of claim 1, wherein the wall is a first wall, the apparatus further comprising:
a second wall coupled to the substrate, the substrate and the second wall defining a lumen in fluid communication with the medicament reservoir,
a distal end portion of the substrate defining a stress concentration riser configured to propagate deformation of the substrate in a predetermined direction when a force is exerted on the distal end portion of the substrate,
a thickness of the second wall is greater than a thickness of the first wall.

7. The apparatus of claim 1, further comprising:
a pipette coupled to the substrate and in fluid communication with the medicament reservoir, the pipette and the wall being monolithically constructed,
a portion of the substrate defining a stress concentration riser configured to propagate deformation of the portion of the substrate and a portion of the pipette in a predetermined direction when a force is exerted on the portion of the substrate such that a lumen defined by the pipette can be placed in fluid communication with a volume outside of the medicament reservoir.

8. The apparatus of claim 7, wherein the wall and the pipette are monolithically constructed.

9. The apparatus of claim 1, wherein the wall is constructed from a substantially inert polymer.

10. The apparatus of claim 1, wherein the medicament reservoir contains fipronil.

11. An apparatus, comprising:
a medicament container including a container portion and a dispensing portion,
the container portion including a wall that defines, at least in part, a medicament reservoir, at least a portion of the wall configured to be deformed to reduce a volume of the medicament reservoir, the wall including a protrusion that extends from the medicament reservoir, the protrusion defining a stress concentration riser configured to propagate deformation of the wall from a predetermined location of the wall,
the dispensing portion configured to place the medicament reservoir in fluid communication with a volume outside of the medicament container.

12. The apparatus of claim 11, wherein the dispensing portion defines a lumen in fluid communication with the medicament reservoir, a portion of the dispensing portion configured to be disrupted to selectively place the medicament reservoir in fluid communication with the volume outside of the medicament container.

13. The apparatus of claim 11, wherein:
the protrusion is disposed at an actuation portion of the wall; and
the portion of the wall is configured to deform at a first rate when the actuation portion is in a first configuration, the portion of the wall configured to deform at a second rate when the actuation portion is in a second configuration.

14. The apparatus of claim 11, wherein:
the protrusion is disposed at an actuation portion of the wall; and
a stiffness of the actuation portion of the wall is different from a stiffness of the portion of the wall.

15. The apparatus of claim 11, wherein:
the dispensing portion defines a lumen in fluid communication with the medicament reservoir, a portion of the dispensing portion configured to be disrupted to selectively place the medicament reservoir in fluid communication with the volume outside of the medicament container;
the wall is tapered such that a cross-sectional area of the medicament reservoir at a distal end portion of the wall is less than a cross-sectional area of the medicament reservoir at a proximal end portion of the wall; and
the proximal end portion of the wall includes the predetermined location.

16. An apparatus, comprising:
a first layer; and
a second layer coupled to the first layer such that the first layer and the second layer define a medicament reservoir, at least a portion of the second layer configured to be deformed to reduce a volume of the medicament reservoir when the second layer is actuated, the second layer being tapered such that a cross-sectional area of the medicament reservoir at a first location along a center line of the medicament reservoir is greater than a cross-sectional area of the medicament reservoir at a second location along the center line,
the second layer including an actuation portion configured to propagate deformation of the second layer from the first location towards the second location.

17. The apparatus of claim, 16 wherein:
the second layer and the first layer collectively define a lumen in fluid communication with the medicament reservoir, a distal end portion of at least one of the first layer or the second layer defining a stress concentration riser configured to propagate a tear in at least one of the first layer or the second layer to place the lumen in fluid communication with an external volume.

18. The apparatus of claim 16, wherein the second layer is configured to deform at a first rate when the actuation portion is in a first configuration, the second layer configured to deform at a second rate when the actuation portion is in a second configuration.

19. The apparatus of claim 16, wherein a stiffness of a first portion of the second layer is different from a stiffness of a second portion of the second layer.

20. The apparatus of claim 16, wherein the actuation portion includes a stress concentration riser.
